(19) United States
(12) Patent Application Publication
(10) Pub. No.: US 2023/0346385 A1
(43) Pub. Date: Nov. 2, 2023

(54) APPARATUS AND METHOD FOR TEMPORARY OCCLUSION OF BLOOD VESSELS

(71) Applicant: Amsel Medical Corporation, Cambridge, MA (US)

(72) Inventors: Arnold Miller, Cambridge, MA (US); Raanan Miller, Cambridge, MA (US); Arthur Z. Bookstein, Chestnut Hill, MA (US)

(73) Assignee: Amsel Medicsl Corporation, Cambridge, MA (US)

(21) Appl. No.: 18/013,995

(22) PCT Filed: Jul. 12, 2021
(86) PCT No.: PCT/US21/41292

§ 371 Date: Jan. 12, 2023

Related U.S. Application Data
(60) Provisional application No. 63/050,099, filed on Jul. 9, 2020.

(51) Int. Cl. A61B 17/12

(52) U.S. Cl. CPC A61B 17/12159; A61B 17/1204; A61B 17/12109; A61B 2017/12004; A61B 2017/12127; A61B 2017/1205

(57) ABSTRACT

The present invention provides temporary occlusion devices and techniques that can be deployed percutaneously to temporarily occlude blood vessels including major blood vessels (e.g., arteries) as well as arteries within organs until specialized care can be obtained to surgically control massive hemorrhage following civilian or military trauma. The temporary occluders of the present invention may be used as an internal tourniquet focused on a specific target region or vessel as an alternative to a conventional tourniquet to control major extremity bleeding following trauma. The temporary occluders of the invention provide a more effective, reliable and highly targeted method to control major blood vessel hemorrhage. Furthermore, unlike a conventional tourniquet, the temporary occluder of the present invention may be used even in the presence of soft tissue injury.

This application relates to methods and devices for temporarily occluding blood vessels, including blood vessels in organs, for example, in situations where one has received an injury and is experiencing significant blood loss.

### BACKGROUND

Uncontrolled hemorrhage remains among most [symbol] cant causes of death in victims who survive a major initial trauma, particularly in truncal and extremity injuries. A loss of 50% of blood volume without replenishment is frequently fatal, and a hypotensive patient, who has lost 30%-35% of blood volume and is in uncompensated shock, is generally close to death. Establishing and maintaining hemostasis at or around the site of an injury is an important consideration in the acute management of trauma patients. The tourniquet, with or without local compression, remains the time-honored method for controlling extremity bleeding following trauma. However, tourniquets generally are useful only for controlling bleeding in limbs, and even then, tourniquets suffer from the disadvantage that they limit blood flow to the entire limb and cannot target individual blood vessels within the limb. It is estimated that of all military wounded who ultimately succumb to their wounds, approximately 10-20% die from blood loss due to inadequate compression or tourniquet application. is a need for more effective temporary blood vessel occlusion techniques and devices for military as well as civilian trauma cases.

It would be desirable to be able to target a specific site in a trauma patient's vasculature at which to place a temporary occlusion device.

### SUMMARY

The present invention provides temporary occlusion devices and techniques that can be deployed percutaneously to temporarily occlude blood vessels including major blood vessels (e.g., arteries) as well as arteries within organs until specialized care can be obtained to surgically control massive hemorrhage following civilian or military trauma. The temporary occluders of the present invention may be used as an internal tourniquet focused on a specific target region or vessel as an alternative to a conventional tourniquet to control major extremity bleeding following trauma. The temporary occluders of the invention provide a more effective, reliable and highly targeted method to control major blood vessel hemorrhage. Furthermore, unlike a conventional tourniquet, the temporary occluder of the present invention may be used even in the presence of soft tissue injury.

In accordance with the invention, blood vessels can be occluded by a percutaneously deliverable occlusion device that includes a pair of cooperating occluder elements. In one embodiment of a two-part occluder, the damaged blood vessel is accessed with a delivery tube, such as a hypodermic needle, that is pre-loaded with the occlusion device. This is within the level of skill of the average first provider such as a military medic or civilian emergency medicaltechnician (EMT). Ultrasound may be used to identify and access a precise target site on the damaged blood vessel where the occluder is to be placed, although other techniques (e.g., tactile sensing) may be employed to sense the presence of a vessel, tubular, or target structure. Ideally, the occluder may be placed upstream of the location of the hemorrhage, although in some instances it may be desirable to also place an occluder at a downstream location to prevent other feeder vessels from directing blood flow to the hemorrhage site. Deployment involves passing a distal occluder element across the blood vessel (e.g., artery) so that the distal element is disposed adjacent or against the outer surface of the blood vessel on the far (distal) side of the blood vessel, and then positioning a proximal occluder element adjacent or against the outer surface of the blood vessel on the near (proximal) side of the blood vessel. The degree of occlusion can be dynamically and controllably varied between full occlusion or partial occlusion by varying the degree of applied compression by adjusting the degree to which the proximal and distal occluder elements are urged toward or away from each other. The device may be provided with a plurality of detents or other temporary mechanism to temporarily maintain the relative positions of the occluders without locking the occluders together. The occluder elements may be maintained in a continually adjustable mode or may be locked together to fully occlude the vessel. Means are provided for unlocking a locked occlusion device should that be desired. In one embodiment, the proximal occluder is removable through the delivery tube. In another embodiment, both of the occluders can be removed through the delivery needle. The occluders can be delivered through a very small delivery tube or needle that leaves a small puncture hole. Thus, following removal, hemostasis of the puncture caused by deployment of the temporary occluder across the blood vessel may be achieved with standard manual compression of the blood vessel, thus minimizing the need for further blood vessel repair. If local compression is not adequate other measures, such as cauterization of the tissue, deploying a polymeric sealant, or deploying gauze or a pad, or positioning a coated stent in the vessel or open surgical repair may be used. Alternatively, other means such as cauterization of the tissue, deploying a polymeric sealant, or deploying gauze or a pad, or positioning a coated stent in the vessel, may be used to arrest blood flow.

### DRAWINGS

The various objects and advantages of the invention will be appreciated more fully from the following description, with reference to the a€companying not-to-scale drawings in which:

FIG. 1 is an illustration of the proximal and distal occluder elements.

FIG. 2 is an illustration of a delivery device for delivering and deploying the occlusion device.

FIG. 3 is an illustration of the distal end of the delivery device as the distal occluder element is delivered.

FIG. 4 is an illustration of the distal end of the delivery device as the proximal occluder element is delivered.

FIG. 5 is an illustration of the distal end of the delivery device when the proximal and distal occluders are brought fully together.

FIG. 6 is an enlarged, diagrammatic illustration of the occlusion device in readiness for deployment, and the elements for delivering the occlusion device, partly in section.

FIG. 7 is an enlarged, diagrammatic illustration of the components of the delivery device when the distal occluder has been partially ejected out the delivery needle.

FIG. 8 is an enlarged, diagrammatic illustration of the components of the delivery device when both the distal and proximal occluders have been deployed pot of the delivery tube.

FIG. 9 is an enlarged, diagrammatic illustration of the components of the delivery device when the occluders have been connected and locked together.

FIG. 10 is an enlarged, diagrammatic illustration of a modified embodiment in which the occluders have been locked together and in which they may be unlocked to enable removal of the proximal occluder.

FIG. 11 is an enlarged, diagrammatic illustration of the modified embodiment of FIG. 10 in which the previously locked occluders have been unlocked.

FIG. 12 is an illustration of the unlocked occluders with the proximal occluder having been partially withdrawn back toward the delivery tube.

FIG. 13 is an illustration of a modified form of occlusion device in which both occlude scan be withdrawn back through the delivery tube.

FIG. 14 is a photographic triptych illustrating the way the occluders may constrain layers in a serpentine configuration.

FIG. 15 is an illustration of a portion of a tube with enhanced flexibility.

### DETAILED DESCRIPTION

FIG. 1 illustrates one embodiment of a two-piece temporary occlusion device that can be inserted percutaneously through tissue to a target site of a blood vessel, upstream and, in some cases, downstream of a hemorrhaging site. The device includes a proximal occluder 10 and a separate distal occluder 12, both shown in their expanded configurations. The occluders are contained, in tandem and in a low-profile configuration, within the lumen of a delivery tube, such as a needle having an outlet opening at its distal end. Each of the proximal and distal occluders 10, 12 is capable of self-expanding from a low-profile configuration in which it can be releasably contained in the needle and a diametrically expanded configuration when it is ejected from the needle. FIG. 6 illustrates the occluders contained in the lumen of a delivery needle, shown in phantom at 14. The occluders can be deployed separately and independently on opposite sides of the traumatized vessel and can be urged together to controllably urge the vessel walls together to fully or partially occlude blood flow and prevent or control blood loss from the damaged vessel.

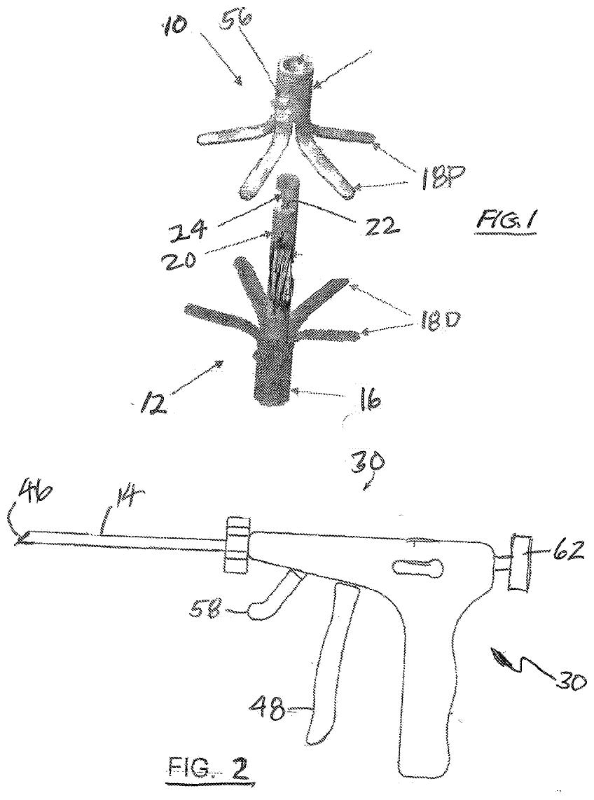

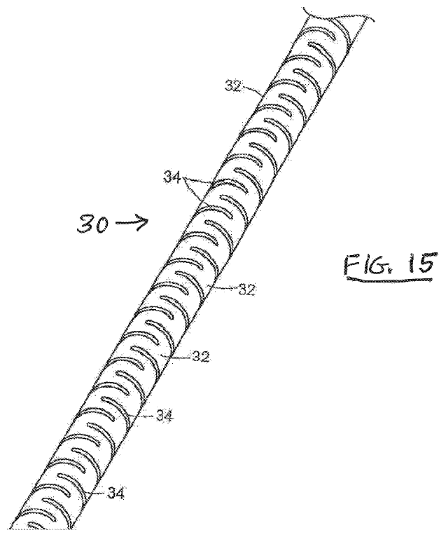

The distal occluder 12 may be formed from a shape memory material such as Nitinol and may have a tubular body 16 with a plurality of longitudinal slits that define a plurality of legs 18D that, when in the deployed, expanded-diameter configuration, extend radially outward from the body 16 as shown in FIG. 1. As shown in FIG. 6, the distal occluder 16 is contained in the needle 14 with its legs constrained in a stressed, low profile configuration in which the legs 18D extend longitudinally in a proximal direction. When released from the delivery tube and unstressed, the legs 18D self-extend radially outward to assume the FIG. 1 configuration. The distal occluder also includes a proximally extending locking post 20 that is attached, at its distal end to the tubular body 16. The proximal end of the post is formed to define one half 22 of a somewhat loose mechanical interlock 24 that is releasably interlocked with a complementary second half 26 of the interlock 24 formed on the distal end of an elongate, distal retention tube 28. The distal retention tube 28 has the same outer diameter as the locking post and extends proximally through the needle 14 to a control handle 30 (FIG. 2) to which it is secured. The distal retention tube 28 may be formed from Nitinol, or other suitable material and, preferably, has at least a portion that is flexible. Its flexibility may be enhanced by having segments 32 (FIG. 15) defined by transverse cuts 34 formed on alternating sides and at spaced intervals along a length of the distal retention tube while also enabling the retention tube 28 to have an adequate degree of column strength as well as tensile strength. Desirably, the portion of the retention tube that is external of the patient should have a flexibility that enables it to be placed in an out-of-the-way location, as by taping it against the patient's skin. The locking post also includes a detent groove 36 that circumscribes the post 20 and cooperates with the proximal occluder 10 to lock the occluders together, as described more fully below.

The proximal occluder 10 similarly may be formed from a shape memory material such as Nitinol and may have a tubular body 38 having an inner diameter dimensioned to slidably fit over the locking post 20 of the distal occluder, and a plurality of distally extending, longitudinal slits that define a plurality of legs 18P that, when in the deployed, expanded-diameter configuration, extend radially outward from the body 16 as shown in FIG. 1. The proximal end of the body 34 is detachably connected to a proximal control tube 42 that, preferably, also may have a flexible portion constructed with transverse slits formed along the length of at least a proximal portion of tube 42, similarly to those on the distal retention tube 28 (FIG. 15), so that itis flexible while also having adequate column, tensile and torsional strength. The proximal occluder 10 is contained in the delivery needle in tandem behind the distal occluder 12 and is formed so that it is contained in the needle 10 with its leg 18P constrained in a low profile and extending distally in readiness to self-deploy to a radially extended configuration when advanced out of the distal end of the needle 14.

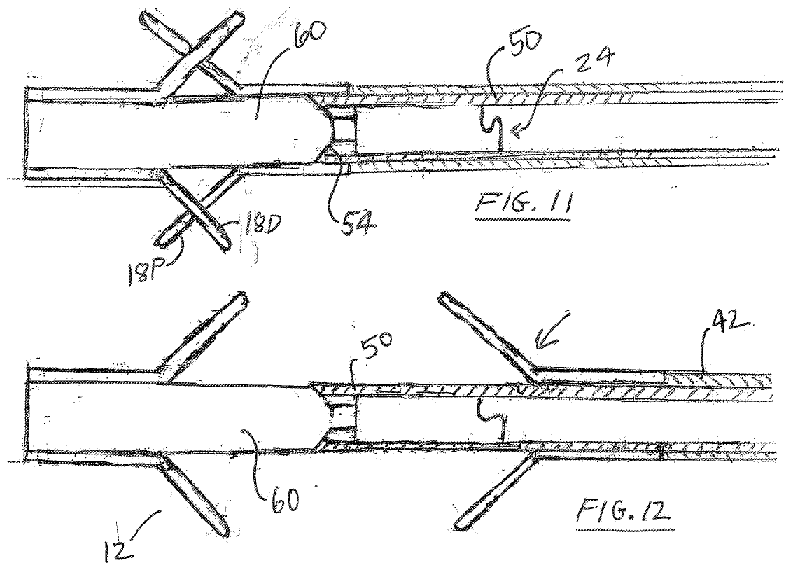

A control handle 30 for controlling the delivery of the occluders is shown in FIG. 2. The delivery needle 14 is attached to the handle and is movable longitudinally of the handle so that it can be retracted to expose, eject, and deploy the occluders. Ultrasound imaging may be employed to facilitate placement of the distal tip of the needle. The control handle is manipulated to advance the needle, containing the occluders 10, 12, percutaneously, through intervening tissue to the target region of the involved vessel where it is passed through the vessel to position the distal outlet 46 of the needle on the far (distal) side of the vessel. With the distal outlet 46 of the needle so positioned, the needle 14 is retracted by operating a first trigger 48 on the handle that is operatively associated with the needle to enable longitudinal needle movement relative to the handle and the occluders within the needle. As the needle is retracted the distal occluder is progressively exposed (as suggested in FIG. 6) until it is fully ejected from the needle, at which time the legs 18D of the distal occluder 12 self-deploy to their expanded configuration (FIGS. 4,7). With the distal occluder deployed and with the proximal occluder still contained in the needle, the proximal occluder can be deployed on the near (proximal) side of the vessel by further retraction of the needle 14 by operating the first trigger 48 to progressively expose the proximal occluder until it is fully ejected, at which time the legs 18P of the proximal occluder assume their expanded configuration on the proximal side of the vessel. With the expanded occluders embracing the vessel, the proximal occluder 10 can be advanced distally, by operating a second trigger 58, which is operatively associated, through the proximal control tube 42, with the proximal occluder 10, to clamp the vessel between the proximal and distal occluders 10, 12. The occluders include a locking mechanism by which they can be locked together, for example, to fully occlude the vessel. The locking mechanism may include a notch 36, such as a detent groove formed on the locking post 20 of the distal ocelude that defines a shoulder 52 at its proximal end and may define a proximally tapering conoid 54 at its distal end. The proximal occluder may include one or more tangs 56 formed in the wall of the tubular body 38, the tang(s) having an unstressed configuration that projects inwardly into the lumen of the body 38 but is pushed back into alignment with the tubular wall of occluder 10 when the proximal occluder is disposed about the outer diameter of the locking post 20 of the distal retention tube 28. As shown in FIG. 9, the proximal occluder may be advanced over the locking post 29 to align the tang 56 with the groove 50 that enables the tang 56 to spring to its unstressed configuration to self-engage the shoulder of the groove 36, thus locking the occluders together in a vessel-clamping configuration. As described in more detail below, means may be provided for unlocking the occluders.

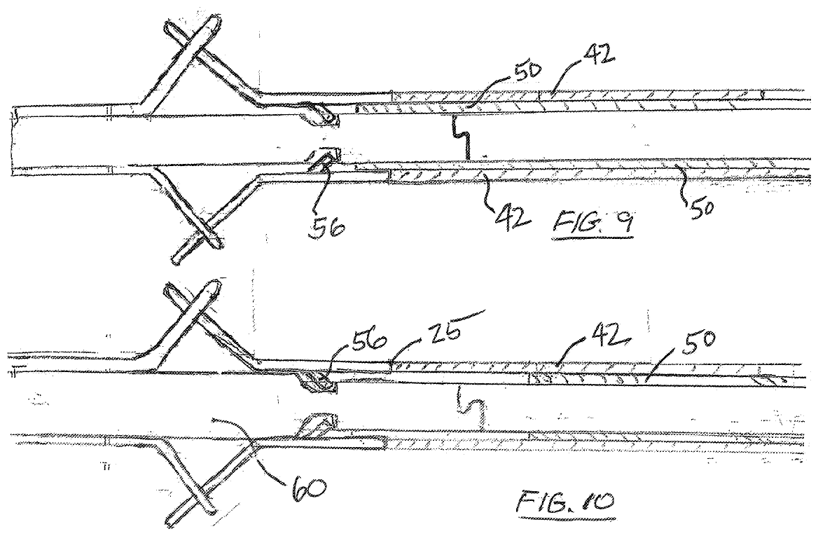

It is advantageous in the case of traumatic injury to a blood vessel, particularly a major blood vessel, to minimize the risk of complete blood stagnation that could lead to clot formation. Thus, the invention enables the medical personnel to control the degree of vessel occlusion by varying intermittently the degree of occlusion applied to the vessel. By varying the extent to which the occluders compress the vessel, the degree of blood flow out of the vessel can be balanced sufficiently with the degree of occlusion to avoid clot formation while minimizing blood loss until the patient can be transported to a facility where the special care to treat the trauma is available. To that end, and to prevent locking of the occluders, the device may be provided with a tubular lock disabling shaft 50 that is slidably disposed about the proximal control tube 42 and the locking tube 20. The disabling shaft 50 extends proximally through the control handle 30 so that its longitudinal position can be controlled by the medical attendant, as by a handle 62 (FIG. 2). FIGS. 6-8 illustrate the device with the disabling tube in a distal position in which it overlies the detent groove 36 of the locking post 20, thus preventing the tang 56 from engaging with the groove 36 while enabling the proximal occluder to slide over the disabling tube 50. Additionally, the disabling tube also serves to overlie the interlock 24 to ensure that the first and second halves 22, 26 of the interlock 24, which are loosely formed to be disengaged by manipulation of the retention tube 28, do not separate inadvertently. Thus, the inner diameter of the disabling tube 50 is such that it can slide closely over the locking tube 20 and the distal retention tube 28. The outer diameter of the disabling tube 50 is slightly less than the inner diameter of the tubular body of the proximal occluder to enable the proximal occluder to slide over the disabling tube 50. With the locking mechanism disabled, the relative positions of the proximal and distal occluders can be variably adjusted to optimally control the degree of occlusion without concern of the occluders becoming locked inadvertently. If, at some point, it is desired to lock the occluders to fully occlude the vessel, the disabling tube 50 can be retracted by handle 62 to expose the groove 36 after which the proximal occluder 10 can be advanced distally by operating the second trigger 58 until the tang 56 engages the groove 36.

In some cases it may be desirable to lock the occluders together and remove the delivery apparatus. That can be achieved by retracting the disabling shaft fromits position overlying the interlock 24 and breaking the connection 25 between the proximal occluder and the proximal control tube. To that end the connection between the proximal occluder and the control shaft may be frangible to permit it to be broken away, such as, for example, by attaching the control tube 28 to the proximal occluder only at a few points as by tack welding, brazing or by other means (threaded connection). Other types of connections may be used to permit the separation.

In some instances it may be desirable to unlock the occluders after they have been locked together. For example, it may be desirable to return to variable adjustment of the degree of occlusion or it may be desirable to remove the proximal occluder by retracting it into the needle. To that end, the device may be modified as shown in FIGS. 10-12, by increasing the diameter of a portion 60 of the locking post immediately below the conoid surface 54 to have an outer diameter substantially equal to that of the disabling tube 50 and so that it can closely slide within the tubular body 38 of the proximal occluder 10. To unlock a previously locked occlusion device, the distal retention tube 28 can be retracted proximally while maintaining the position of the proximal occluder, thus advancing the distal occluder and its locking post 20 proximally relative to the proximal occluder. That causes the conoid portion 54 of the groove 36 to engage the tang 56 to urge the tang radially outward into alignment with the wall of the proximal occluder body, thus disengaging those locking elements. Then, as shown in FIG. 12, the disabling tube 50 may be advanced distally to locate its distal end adjacent the outermost portion of the conoid 54 so that the disabling tube 50 and post portion 60 present a continuous outer diameter over which the proximal occluder may retracted, either to reestablish blood flow control or to entirely remove the proximal occluder 10 as suggested in FIG. 12. It may be noted that the slight retraction of the retention tube 28 will tend to increase the force applied to the tissue. The unaligned legs 18P, 18D of the occluders, however, are relatively resilient and may yield without applying excessive force to the tissue.

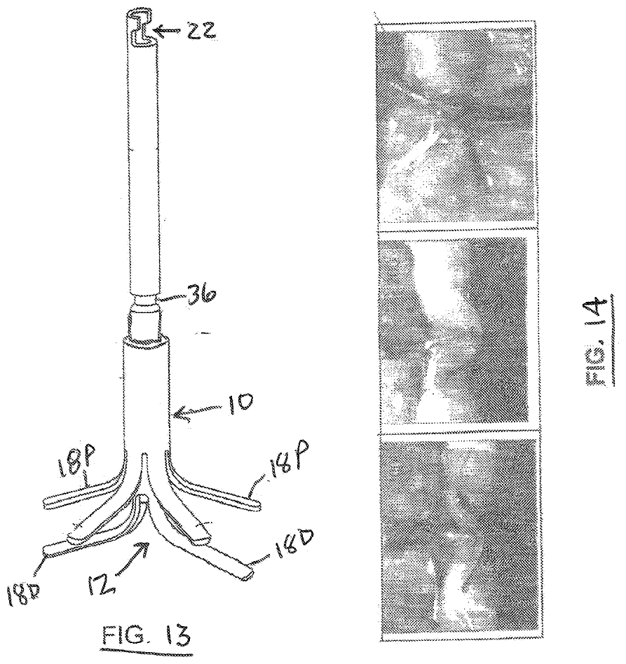

It may be noted that in the embodiments described above, the legs 18P, 18D, when in their expanded configurations, extend at an angle from their respective bodies that define a concave, conoid configuration in which the concavities of the occluders face each other. FIG. 13 illustrates a modified embodiment of the invention in which the legs 18P, 18D of both occluders, when in their expanded configurations, define concavities that face in a distal direction. With this embodiment, if desired, it is possible to withdraw both occluders back into the delivery tube or needle, with the legs being urged radially inwardly as the occluders are progressively withdrawn through the distal outlet 46 of the needle.

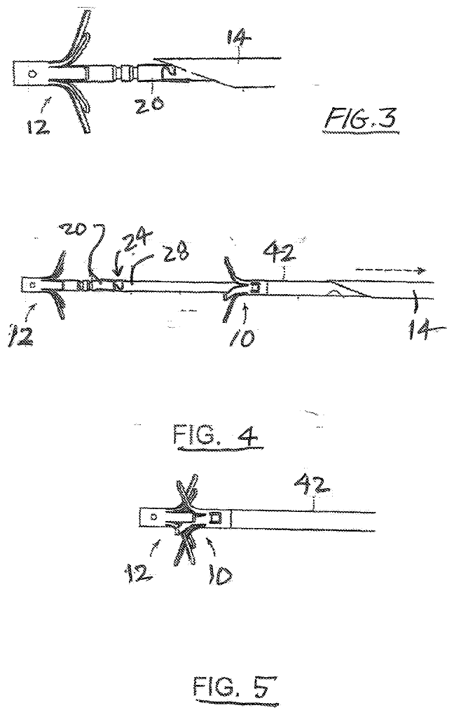

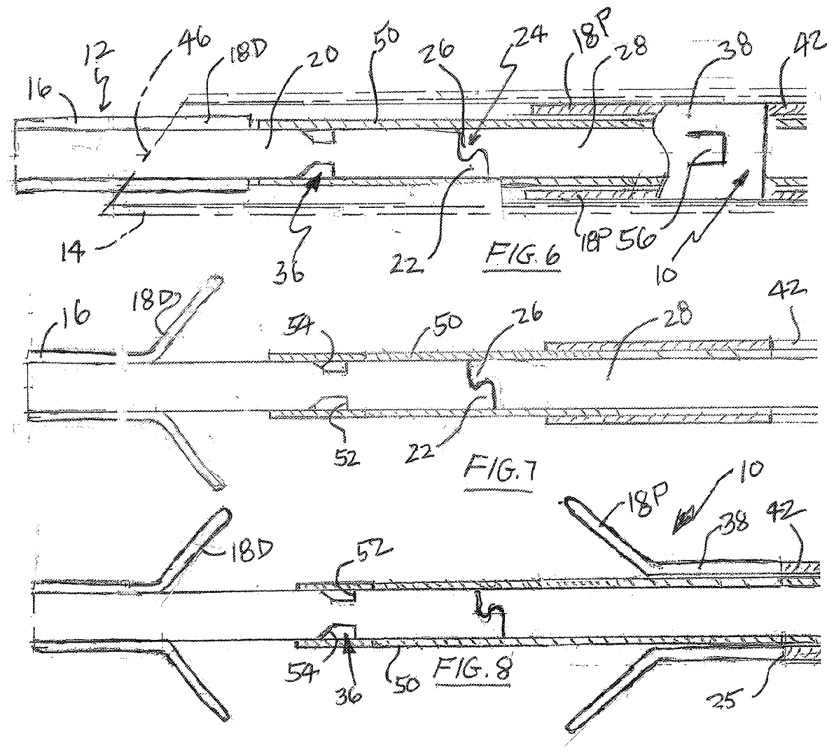

Also, among the advantageous features of the invention is the way the proximal and distal occluders may cooperate to occlude the vessel in a relatively atraumatic manner that reduces the risk of further damage to the vessel. The occluders are configured so that when they are deployed, they can be oriented so that the legs 18P, 18D of the proximal and distal occluders 10, 12 are out of registry with each other. FIG. 14 illustrates the arrangement of the legs 18P. 18D of the proximal and distal implants in which the out-of-registry legs are sufficiently interdigitated so that they do not effect a direct compressive clamping of the tissue but, instead, engage the tissue to constrain the tissue in a serpentine configuration extending at least partly about the axis of the occluder in a generally circumferential direction. In this arrangement, the legs of one of the implants are out of registry with those of the other implant so that when viewed in plan, the legs of one implant lie between the legs of the other. In this configuration the opposing walls of the vessel are together partially wrapped about the legs 18P, 18D in alternating directions to constrain the tissue in a serpentine configuration as seen diagrammatically in FIG. 14. FIG. 5 illustrates the interdigitation of the occluder legs in the absence of tissue. Even if the thickness or other characteristic of the clamped vessel is such that the occluder legs do not actually achieve interdigitation, the legs nonetheless apply oppositely directed forces at circumferentially spaced locations about the axis of the occluders the clamped tissue layer. Such application of clamping force is less likely to cause additional tissue injury than direct clamping. The legs of the occluders are not plastically formed but are in an elastic state to act as springs that can adjust to the tissue properties such as thickness, stiffness, etc. while still providing sufficient force to compress the vessel or tissue without crushing the tissue and causing further damage. The legs of one or both occluders may be formed to extend at an acute angle to the occluder axis to define a conical configuration. This provides an additional means by which the clamping forces can be adjustably controlled. By selecting, during manufacture, a particular cone angle defined by the expanded legs as well as their stiffness, the characteristics of the serpentine pattern can be varied.

In the illustrative embodiment of FIG. 6, the entire apparatus can be contained in a hollow needle or delivery tube with an outer diameter of less than 1.7 mm or less than 1.5 mm. The proximal control tube 28 may have an outer diameter of about 0.7 mm; the outer diameter of tube 42 may be about 1.3 mm with an inner diameter of about 1.0 mm; the inner diameter of tube 50 is just slightly larger than 0.7 mm with an outer diameter of just slightly smaller than 1 mm. The depth of the locking region 36 may be about 0.1 mm. This results in a relatively small puncture hole in the vessel that is easier to close upon removal of the delivery device which may be closable simply by application of local pressure.

FIG. 14 shows three photographs of serpentine folds formed in a simulated blood vessel. Those folds or ripples extend radially beyond the ends of the legs 18P, 18D, effectively extending the radial extent of the closure and defining a circumferential band with a diameter greater than that of the occluders. By way of example but not limitation, a two-part occluder as described above and having legs that define a diameter of 5.5 mm may be able to occlude vessels that are over 7 mm (and even equal or greater than 1 cm) in diameter. The ability of the two-occluder to cause serpentine ripples beyond the diameter of the legs 18 of the device enables the device to be at least partly effective and, possibly fully effective, even if the needle misses and does not transfix the vessel but is sufficiently close to the vessel so that some of the legs, or at least some of the circumferential band defined by the serpentine ripples overlies at least a portion, and possibly all, of the width of the vessel. The invention thus provides a margin of error and may enable sufficient temporary occlusion to be achieved even if the target vessel is not transfixed by the needle.

The foregoing description of the invention is intended merely to be illustrative thereof and that various modifications and equivalents may be apparent to those skilled in the art without departing from its principles

1. An apparatus for temporarily controlling blood loss from a blood vessel comprising:
a delivery tube having a distal outlet;
a distal occluder contained in the delivery tube and comprising a distal body and a plurality of legs extending from the distal body and which may assume (i) a diametrically reduced delivery configuration in which it can be contained in the delivery tube and (ii) a diametrically expanded deployed configuration in which the legs are extended radially of the distal body;
a proximal occluder contained in the delivery tube proximally of and separate from the distal occluder, the proximal occluder comprising a proximal body and a plurality of legs extending from the body, the legs being configured to assume (i) a diametrically reduced delivery configuration in which it can be contained in the delivery tube and (ii) a diametrically expanded deployed configuration in which the legs are extended radially of the proximal body;
the proximal and distal occluders, when deployed, being adjustably movable toward and away from each other thereby controllably varying the degree to which a blood vessel disposed between the occluders can be occluded:
each occluder, when deployed, having a portion that is connectible with a portion of the other occluder, each of the connectible portions having a locking element engageable with a locking element of the other occluder, thereby to lock the deployed occluders together; and
a disabling member insertable between the connectible portions of the occluders to selectively prevent or permit engagement of the locking elements with each other.

2. The apparatus as defined in claim 1 further comprising:
the legs of the occluders being interdigitated when the occluders are locked together in the absence of tissue between the occluders.

3. The apparatus as defined in claim 1 further comprising:
a handle at the proximal end of the apparatus, the delivery tube being mounted to the handle for movement in a proximal direction from a distal position;
the distal occluder having an integral, proximally extending locking post:
a distal retention shaft fixed to the handle and detachably connected to the proximal end of the locking post, thereby to maintain the relative position of the distal occluder during retraction of the delivery tube from its distal position;
the locking post comprising the portion of the distal occluder that is connectible to the proximal occluder.

4. The apparatus as defined in claim 3 further comprising:
a proximal control shaft having proximal and distal ends, the distal end being connected to the proximal occluder, the distal end being operatively associated with a control on the handle for moving the proximal occluder toward and away from the distal occluder.

5. The apparatus as defined in claim 4 wherein the connection between the proximal control shaft and the proximal occluder is separable by manipulation from the handle.

6. The apparatus as defined in claim 3 wherein the locking element of the distal occluder comprises a detent notch formed on the locking post.

7. The apparatus as defined in claim 6 further comprises:
the proximal body of the proximal occluder is tubular and comprises the connectible portion of the proximal occluder and is receptive to the locking post;
at least one tang formed from the tubular proximal body, the tang having an unstressed configuration that projects radially inwardly of the tubular proximal body, the tang having sufficient resilience to be urged radially outwardly to a stressed position from its unstressed position;
the tang, when in its unstressed configuration, being engageable with the detent notch of the locking post, thereby locking the occluders together.

8. The apparatus as defined in claim 7 wherein the disabling member comprises:
a disabling tube disposed between the locking post and proximal body of the proximal occluder and being movable between a distal position in which it blocks engagement of the locking elements and a retracted position in which the engagement of the locking elements is enabled.

9. The apparatus as defined in claim 8 further comprising:
the locking post having a proximal portion proximally of the notch and a distal portion distal of the notch, the proximal portion being of a diameter receivable by the tubular proximal body, the distal portion having an outer diameter substantially the same as the disabling tube:
whereby locked occluders can be unlocked by advancing the distal occluder and locking post proximally to cause the distal portion of locking post may engage and urge the tang outwardly to its stressed position, disengaged from the notch.

10. The apparatus as defined in claim 1 wherein the delivery tube comprises a needle having a sharp, tissue piercing tip.

11. The apparatus of claim 1 wherein the disabling member is movable between positions that, selectively, enable or disable engagement of the locking elements.

12. The apparatus as defined in claim 4 wherein the disabling member overlies the connection between the locking post and retention shaft to prevent said separation.

13. The apparatus as defined in claim 12 wherein the disabling member is retractable to a non-overlying position to enable separation of the locking post and distal retention shaft.

14. The apparatus as defined in claim 1 further comprising:
the legs of the proximal occluder being out of registry with the legs of the distal occluder so that when a vessel is clamped by the occluders, the legs will apply oppositely directed forces to the vessel at circumferentially spaced locations about an axis of the occluders.

15. The apparatus as defined in claim 14 wherein the occluder have sufficient stiffness to constrain the vessel walls in a serpentine configuration.

16. The apparatus as defined in claim 1 wherein the connectible portions of the occluders are telescopically connectible.
